(19) United States
(12) Patent Application Publication
(10) Pub. No.: US 2008/0206153 A1
(43) Pub. Date: Aug. 28, 2008

(54) Copolymers for Cosmetic Agents, Produced in the Presence of Polyfunctional Chain Transfer Agents

(75) Inventors: Gabi Winter, Ludwigshafen (DE); Lysander Chrisstoffels, Limburgerhof (DE); Claudia Wood, Weinheim (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 11/814,448

(22) PCT Filed: Jan. 20, 2006
(86) PCT No.: PCT/EP06/00516

§ 371 Date: Jul. 20, 2007

(30) Foreign Application Priority Data
Jan. 21, 2005 (DE) 102005003010.6

(51) Int. Cl. A61K 8/81; A61K 47/32; A61K 9/12; C08F 2/38

(52) U.S. Cl. 424/43; 514/772.4; 514/772.6; 424/70.11; 514/772.1; 524/555; 524/556; 524/548

(57) ABSTRACT

The present invention relates to cosmetic and pharmaceutical compositions which comprise a copolymer with anionogenic and/or anionic groups which is obtainable by free-radical polymerization of a monomer mixture in the presence of a polyfunctional regulator with at least three functional regulating groups. The invention further relates to a method of producing such a copolymer and to the copolymers obtainable by this method.

The present invention relates to cosmetic and pharmaceutical compositions which comprise a copolymer with anionogenic and/or anionic groups and which is obtainable by free-radical polymerization of a monomer mixture in the presence of a polyfunctional regulator with at least three functional regulating groups. The invention further relates to a method of producing such a copolymer and to the copolymers obtainable by this method.

Cosmetically and pharmaceutically acceptable water-soluble or water-dispersible polymers are used widely in cosmetics and medicine. They are used, for example, quite generally as film formers and thickeners for diverse types of formulations, such as sprays, gels, creams, etc. For these applications, use is often made of branched or crosslinked water-soluble polymers with anionic functionalities such as, for example, crosslinked polyacrylic acid. For hair cosmetics in particular, polymers with film-forming properties are used as setting agents and conditioners in order to impart hold to the hairstyle, and to improve the dry and wet compatibility, the feel to the touch, the shine and/or the appearance of the hair, and to impart antistatic properties to the hair. Besides the abovementioned carboxylate group-containing polymers, the conditioners used are often crosslinked polymers with cationic functionalities which have a high affinity to the surface of the hair, which is negatively charged as a result of its structure. These include, for example, crosslinked copolymers of N-vinylpyrrolidone, quaternized N-vinylimidazole, acrylamide and diallyldimethylammonium chloride (DADMAC).

Difficulties often arise with the provision of products with a complex profile of properties. Thus, for example, there is a need for polymers for cosmetic and pharmaceutical compositions which firstly have good film-forming properties and secondly can also be formulated and applied easily. Besides the film-forming properties, the rheological properties play an important role here. There thus continues to be a lack of polymers for use in hair sprays which, as well as having good film-forming properties, are suitable for producing formulations with the lowest possible viscosity. Such polymers would then also be suitable for use in other fields of application, for example in pharmacy or in the agriculture sector.

WO 94/24986 describes hair-setting compositions which comprise copolymers based on tert-butyl acrylate or tert-butyl methacrylate as film formers. During the preparation of these copolymers it is possible to use regulators, for example sulfur compounds, for example mercaptoethanol.

WO 2004/058837 describes an ampholytic copolymer which is obtainable by free-radical copolymerization of
a) at least one ethylenically unsaturated compound with at least one anionogenic and/or anionic group,
b) at least one ethylenically unsaturated compound with at least one cationogenic and/or cationic group,
c) at least one unsaturated amide group-containing compound
   and if appropriate further comonomers. For producing these ampholytic copolymers, the use of regulators is also quite generally described.

WO 2004/022616 describes hair cosmetic preparations based on cationogenic/cationic polymers which are obtainable by
(i) free-radically initiated copolymerization of monomer mixtures of
   (a) at least one cationic monomer or quaternizable monomer,
   (b) if appropriate a water-soluble monomer,
   (c) if appropriate a further free-radically copolymerizable monomer,
   (d) at least one monomer acting as crosslinker having at least two ethylenically unsaturated nonconjugated double bonds, and
   (e) at least one regulator,
(ii) subsequent quaternization or protonation of the polymers if an unquaternized or only partially quaternized monomer is used as monomer (a).

WO 2004/058831 describes an aqueous dispersion obtainable by free-radical polymerization of
a) at least one N-vinyl-containing monomer,
b) at least one polymeric dispersant,
c) at least one polymeric precipitation agent,
d) at least one crosslinker,
e) if appropriate further monomers,
f) if appropriate at least one regulator,
g) if appropriate a buffer substance,
   where the weight ratio of b) to c) is in the range from 1:50 to 1:0.02. Also disclosed is the use of this dispersion in cosmetic preparations.

The unpublished German patent application P 103 31 865.8 describes an aqueous polymer dispersion Pd) obtainable by free-radical polymerization of a monomer mixture M) comprising
a) at least one α,β-ethylenically unsaturated amide group-containing compound of the general formula I

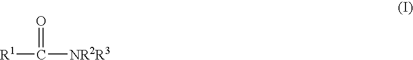

where
   R² is a group of the formula CH₂═CR⁴ and R¹ and R³, independently of one another, are H, alkyl, cycloalkyl, heterocycloalkyl, aryl or hetaryl, or R¹ and R³, together with the amide group to which they are bonded, are a lactam with 5 to 8 ring atoms,
b) at least one free-radically polymerizable crosslinking compound with at least two α,β-ethylenically unsaturated double bonds per molecule,
c) at least one compound with a free-radically polymerizable α,β-ethylenically unsaturated double bond and at least one cationogenic and/or cationic group per molecule,
   in an aqueous medium in the presence of at least one polymeric anionic dispersant D). Also described are cosmetic or pharmaceutical compositions which comprise such a polymer dispersion or a polymer obtainable by drying such a dispersion. For producing this polymer dispersion, the use of mono-, di- and polyfunctional regulators is also quite generally described.

WO 02/38638 describes acrylate polymers based on tert-butyl acrylate and/or tert-butyl methacrylate which are obtained by free-radical polymerization in the presence of C₁₄-C₂₂-alkanethiols. They are characterized by their neutral odor and are suitable for cosmetic formulations which comprise no additional fragrances. The use of polyfunctional regulators for producing these acrylate polymers is not described.

There is still a need for polymers which have good film-forming properties and which at the same time are suitable for producing formulations with the lowest possible viscosity. The object of the present invention is to provide such polymers. These should specifically be suitable for producing compositions for setting the hair, improving the structure of the hair and/or shaping the hair. Preferably, they should have two or more of the following properties: efficient hair setting, good sprayability, good ability to be washed out and the ability to formulate low voc formulations.

Surprisingly, it has now been found that this object is achieved by copolymers which comprise anionogenic and/or anionic groups and which are obtainable by free-radical polymerization in the presence of a polyfunctional regulator with at least three functional regulating groups.

The invention therefore provides a cosmetic or pharmaceutical composition comprising
A) at least one copolymer with anionogenic and/or anionic groups obtainable by free-radical polymerization of a monomer mixture M) comprising
   a) at least one compound with a free-radically polymerizable α,β-ethylenically unsaturated double bond and at least one anionogenic and/or anionic group per molecule, and
   b) at least one monomer which is chosen from esters of α,β-ethylenically unsaturated mono- and dicarboxylic acids with C₁-C₃₀-alkanols, N-alkyl- and N,N-dialkylamides, α,β-ethylenically unsaturated monocarboxylic acids, and mixtures thereof,
in the presence of
   c) at least one compound which has a regulating effect on the molecular weight of the resulting polymers and which has at least 3 functional regulating groups (polyfunctional regulator),
B) at least one cosmetically or pharmaceutically acceptable carrier.

The invention further provides a method of producing copolymers A) with anionogenic and/or anionic groups by free-radical polymerization of a monomer mixture M) comprising monomers a), b) and if appropriate further monomers, in the presence of at least one polyfunctional regulator c). The invention further provides the copolymers A) obtainable by this method.

It has been found that the copolymers A) according to the invention have very good film-forming properties and are also suitable for producing formulations with a low viscosity. They have better application properties than corresponding polymers with a low molecular weight known from the prior art. Thus, for a comparable setting effect, they exhibit significantly improved rheological properties compared with polymers of identical composition which have been produced in the presence of mono- or difunctional regulators. This manifests itself, for example, in improved sprayability. The copolymers A) firstly have a high propellant gas compatibility, and are secondly also advantageously suitable for producing formulations with a low content of volatile organic compounds, i.e. so-called low voc formulations (voc=volatile organic compounds).

For the purposes of the present invention, the expression alkyl comprises straight-chain and branched alkyl groups. Suitable short-chain alkyl groups are, for example, straight-chain or branched C₁-C₇-alkyl groups, preferably C₁-C₆-alkyl groups and particularly preferably C₁-C₄-alkyl groups. These include, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl etc.

Suitable longer-chain C₈-C₃₀-alkyl and C₈-C₃₀-alkenyl groups are straight-chain and branched alkyl and alkenyl groups. These are preferably predominantly linear alkyl radicals, as also arise in natural or synthetic fatty acids and fatty alcohols and also oxo alcohols, which may if appropriate additionally be mono-, di- or polyunsaturated. These include, for example, n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene) etc.

Cycloalkyl is preferably C₅-C₈-cycloalkyl, such as cyclopentyl, cyclohexyl, cycloheptyl or cyclooctyl.

Aryl comprises unsubstituted and substituted aryl groups and is preferably phenyl, tolyl, xylyl, mesityl, naphthyl, fluorenyl, anthracenyl, phenanthrenyl, naphthacenyl and in particular phenyl, tolyl, xylyl or mesityl.

Hereinafter, compounds which can derive from acrylic acid and methacrylic acid can sometimes be referred to in short by adding the syllable “(meth)” to the compound derived from acrylic acid.

Within the scope of the present invention, water-soluble monomers and polymers are understood as meaning monomers and polymers which dissolve in water in an amount of at least 1 g/l at 20° C. Water-dispersible monomers and polymers are understood as meaning monomers and polymers which disintegrate into dispersible particles under the application of shear forces, for example by stirring. The copolymers used according to the invention are generally water-soluble or at least water-dispersible.

Monomer a)

The monomer mixture M) comprises, as compound a), at least one compound with a free-radically polymerizable α,β-ethylenically unsaturated double bond and with at least one anionogenic and/or anionic group per molecule. Component a) is preferably used in an amount of from 0.1 to 99.89% by weight, particularly preferably 0.5 to 70% by weight, in particular 10 to 40% by weight, specifically 15 to 30% by weight, based on the total weight of the components used for the polymerization (i.e. components a), b), c) and, if present, d) to g)). The compounds of component a) are generally water-soluble monomers.

The compounds a) are preferably chosen from monoethylenically unsaturated carboxylic acids, sulfonic acids, phosphonic acids and mixtures thereof.

The monomers a) include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers a) further include the half-esters of monoethylenically unsaturated dicarboxylic acids having 4 to 10, preferably 4 to 6, carbon atoms, e.g. of maleic acid, such as monomethyl maleate. The monomers a) also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid. The monomers a) also include the salts of the abovementioned acids, in particular the sodium, potassium and ammonium salts, and the salts with amines. The monomers a) can be used as they are or as mixtures with one another. The stated weight fractions all refer to the acid form.

Component a) is preferably chosen from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof.

Component a) is particularly preferably chosen from acrylic acid, methacrylic acid, itaconic acid and mixtures thereof.

Monomer b)

Component b) is preferably used in an amount of from 0.1 to 99.89% by weight, particularly preferably 1 to 99% by weight, in particular 60 to 90% by weight, based on the total weight of the components used for the polymerization (i.e. components a), b), c) and, if present, d) to g)). An especially suitable quantitative range for component b) is 70 to 85% by weight. The compounds of component b) are generally water-insoluble monomers.

Suitable monomers b) are methyl(meth)acrylate, methyl ethacrylate, ethyl(meth)acrylate, ethyl ethacrylate, tert-butyl(meth)acrylate, tert-butyl ethacrylate, n-octyl(meth)acrylate, 1,1,3,3-tetramethylbutyl(meth)acrylate, ethylhexyl(meth)acrylate, n-nonyl(meth)acrylate, n-decyl(meth)acrylate, n-undecyl(meth)acrylate, tridecyl(meth)acrylate, myristyl(meth)acrylate, pentadecyl(meth)acrylate, palmityl(meth)acrylate, heptadecyl(meth)acrylate, nonadecyl(meth)acrylate, arachinyl(meth)acrylate, behenyl(meth)acrylate, lignoceryl(meth)acrylate, cerotinyl(meth)acrylate, melissinyl(meth)acrylate, palmitoleinyl(meth)acrylate, oleyl(meth)acrylate, linolyl(meth)acrylate, linolenyl(meth)acrylate, stearyl(meth)acrylate, lauryl(meth)acrylate and mixtures thereof.

Suitable monomers b) are also N-methyl(meth)acrylamide, N-ethyl(meth)acrylamide, N-propyl(meth)acrylamide, N-(n-butyl)(meth)acrylamide, N-(tert-butyl)(meth)acrylamide, N-(n-octyl)(meth)acrylamide, N-(1,1,3,3-tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-arachinyl(meth)acrylamide, N-behenyl(meth)acrylamide, N-lignoceryl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide, N-lauryl(meth)acrylamide, N,N-dimethyl(meth)acrylamide, N,N-diethyl(meth)acrylamide, morpholinyl(meth)acrylamide and mixtures thereof.

Polyfunctional Regulator c)

According to the invention, the free-radical polymerization of the monomer mixture M) takes place in the presence of at least one polyfunctional regulator c). The regulators c) are preferably used in a feed amount of from 0.01 to 10% by weight, particularly preferably from 0.05 to 5% by weight and in particular from 0.1 to 2.5% by weight, based on the total weight of the components used for the polymerization (i.e. components a), b), c) and, if present, d) to g)).

Regulators (polymerization regulators) is generally the term used for compounds with high transfer constants. Regulators accelerate chain-transfer reactions and thus bring about a reduction in the degree of polymerization of the resulting polymers without influencing the gross reaction rate.

Within the scope of the present invention, a distinction is made between mono-, bi- or polyfunctional regulators depending on the number of functional groups in the molecule which can lead to one or more chain-transfer reactions.

Suitable polyfunctional regulators are compounds which comprise more than two halogen atoms in bonded form. Examples thereof are alkyl halides, such as tetrachloromethane, chloroform, bromotrichloromethane and bromoform.

Suitable polyfunctional regulators are compounds which comprise more than two sulfur atoms in bonded form. Examples thereof are trifunctional, tetrafunctional and polyfunctional mercaptans. These include the polyvalent sulfur compounds described in EP-A-1 086 980, which is hereby incorporated in its entirety by reference. The regulators preferably used are, for example, esters of ω-mercaptomonocarboxylic acids with trihydric and polyhydric alcohols. Suitable ω-mercaptomonocarboxylic acids are, for example, 2-mercaptoacetic acid (thioglycolic acid), 3-mercaptopropionic acid, 4-mercaptobutyric acid, 5-mercaptopentanoic acid, 6-mercaptohexanoic acid, etc. Suitable alcohols are, for example, glycerol, trimethylolpropane, erythritol, pentaerythritol, dipentaerythritol, etc.

Preferred polyfunctional regulators are polyfunctional mercaptans, e.g. of the formulae

(HS(CH₂)ₙCO₂CH₂)₃C—R

(HS(CH₂)ₙCO₂CH₂)₄C

and

(HS(CH₂)ₙCO₂CH₂)₂CH(O₂C(CH₂)ₙSH)

in which n is an integer from 1 to 10 and R is C₁-C₄-alkyl.

Preferred trifunctional mercaptans are
1,1,1-tris(hydroxymethyl)ethane tris(2-mercaptoacetate)
(=1,1,1-trimethylolethan-tris(2-mercaptoacetate)
(=1,1,1-tris(2-mercaptoacetoxymethyl)ethane),
1,1,1-tris(hydroxymethyl)ethane tris(3-mercaptopropionate),
1,1,1-tris(hydroxymethyl)ethane tris(4-mercaptobutyrate),
1,1,1-tris(hydroxymethyl)ethane tris(5-mercaptopentanoate),
1,1,1-tris(hydroxymethyl)ethane tris(6-mercaptohexanoate),
1,1,1-tris(hydroxymethyl) propane tris(2-mercaptoacetate),
1,1,1-tris(hydroxymethyl)propane tris(3-mercaptopropionate),
1,1,1-tris(hydroxymethyl)propane tris(4-mercaptobutyrate),
1,1,1-tris(hydroxymethyl)propane tris(5-mercaptopentanoate),
1,1,1-tris(hydroxymethyl)propane tris(6-mercaptohexanoate),
glycerol tris(2-mercaptoacetate),
glycerol tris(3-mercaptopropionate),
glycerol tris(4-mercaptobutyrate),
glycerol tris(5-mercaptopentanoate),
glycerol tris(6-mercaptohexanoate),
trithiocyanuric acid,
tris(2-(3-mercaptopropionyloxy)ethyl)isocyanurate.

Preferred tetrafunctional mercaptans are
pentaerythritol tetrakis(2-mercaptoacetate)
pentaerythritol tetrakis(3-mercaptopropionate),
pentaerythritol tetrakis(4-mercaptobutyrate),
pentaerythritol tetrakis(5-mercaptopentanoate)
pentaerythritol tetrakis(6-mercaptohexanoate) and
tetrathiopentaerythritol.

Preferred hexafunctional mercaptans are
dipentaerythritol hexakis(2-mercaptoacetate) and
dipentaerythritol hexakis(3-mercaptopropionate).

For use as regulators, particularly preferred polyvalent sulfur compounds are:
1,1,1-tris(hydroxymethyl)ethane tris(2-mercaptoacetate),
1,1,1-tris(hydroxymethyl)ethane tris(3-mercaptopropionate)
1,1,1-tris(hydroxymethyl)propane tris(2-mercaptoacetate),
1,1,1-tris(hydroxymethyl)propane tris(3-mercaptopropionate),
pentaerythritol tetrakis(2-mercaptoacetate),
pentaerythritol tetrakis(3-mercaptopropionate),
glycerol tris(2-mercaptoacetate),
glycerol tris(3-mercaptopropionate),
dipentaerythritol hexakis(3-mercaptopropionate),
trithiocyanuric acid,
tris(2-(3-mercaptopropionyloxy)ethyl) isocyanurate and
tetrathiopentaerythritol.

Suitable further polyfunctional regulators are Si compounds, as described in DE-A-102 37 378. These include oligomers based on compounds of the formula Ia and with structural elements of the formula Ib

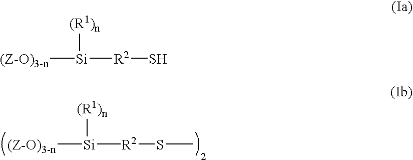

where, in the formulae Ia and Ib
n is an integer from 0 to 2,
R¹ is C₁-C₁₈-alkyl or aryl, preferably phenyl, where if n=2 the radicals R¹ may have identical or different meanings,
R² is C₁-C₁₈-alkylene, cycloalkylene, preferably cyclohexylene, or arylene, preferably phenylene,
Z is C₁-C₁₈-alkyl, C₂-C₁₈-alkenyl or C₂-C₁₈-alkynyl, where one or more nonadjacent carbon atoms may also be replaced by a heteroatom or a heteroatom-containing group chosen from O, S, NR^{a} or SiR^{b}R^{c}, in which R^{a} is hydrogen, alkyl, cycloalkyl or aryl and R^{b} and R^{c}, independently of one another, are alkyl, cycloalkyl or aryl, or
Z is one of the groups

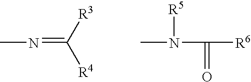

in which
R³, R⁴, R⁵ and R⁶, independently of one another, are alkyl, cycloalkyl or aryl.

All of the specified polyfunctional regulators can be used individually or in combination with one another.

Cationogenic/Cationic Monomer d)

According to the invention, the monomer mixture M) used to produce the copolymers has monomers with anionogenic and/or anionic groups. In a suitable embodiment, for the polymerization it is additionally possible to also use monomers with cationogenic and/or cationic groups (monomer d)). The amount of monomers with cationogenic and cationic groups used for the polymerization is such that, based on the monomers used overall for the polymerization, the mole fraction of cationogenic and cationic groups is less than the mole fraction of anionogenic and anionic groups. The copolymers therefore have on average a molar excess of anionogenic/anionic groups compared with cationogenic/cationic groups.

Component d) is preferably used in an amount of from 0 to 50% by weight, particularly preferably 0 to 40% by weight, in particular 0 to 30% by weight, based on the total weight of the components used for the polymerization (i.e. components a), b), c) and, if present, d) to g)). If a component d) is used, then it is preferably in an amount of at least 0.1% by weight, particularly preferably at least 1% by weight, in particular at least 5% by weight. A particularly preferred quantitative range is 5 to 15% by weight.

Suitable monomers d) have a free-radically polymerizable α,β-ethylenically unsaturated double bond and additionally at least one cationogenic and/or cationic group per molecule. The cationogenic and cationic groups of component d) are preferably nitrogen-containing groups, such as primary, secondary and tertiary amino groups, and quaternary ammonium groups. The nitrogen-containing groups are preferably tertiary amino groups or quaternary ammonium groups. Charged cationic groups can be produced from the amine nitrogens either by protonation, e.g. with monobasic or polybasic carboxylic acids, such as lactic acid or tartaric acid, or mineral acids, such as phosphoric acid, sulfuric acid and hydrochloric acid, or by quaternization, e.g. with alkylating agents, such as C₁-C₄-alkyl halides or sulfates. Examples of such alkylating agents are ethyl chloride, ethyl bromide, methyl chloride, methyl bromide, dimethyl sulfate and diethyl sulfate.

Suitable compounds d) are, for example, the esters of α,β-ethylenically unsaturated mono- and dicarboxylic acids with aminoalcohols. Preferred aminoalcohols are C₂-C₁₂-aminoalcohols which are C₁-C₈-dialkylated on the amine nitrogen. Suitable acid components of these esters are, for example, acrylic acid, methacrylic acid, fumaric acid, maleic acid, itaconic acid, crotonic acid, maleic anhydride, monobutyl maleate and mixtures thereof. Preference is given to using acrylic acid, methacrylic acid and mixtures thereof. Preference is given to N,N-dimethylaminomethyl(meth)acrylate, N,N-dimethylaminoethyl(meth)acrylate, N,N-diethylaminoethyl(meth)acrylate, N,N-dimethylaminopropyl(meth)acrylate, N,N-diethylaminopropyl(meth)acrylate and N,N-dimethylaminocyclohexyl(meth)acrylate.

Suitable monomers d) are also the amides of the abovementioned α,β-ethylenically unsaturated mono- and dicarboxylic acids with diamines which have at least one primary or secondary amino group. Preference is given to diamines which have a tertiary and a primary or secondary amino group. As monomers d), preference is given to using N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide, N-[4-(dimethylamino)cyclohexyl]methacrylamide etc.

Suitable monomers d) are also N,N-diallylamines and N,N-diallyl-N-alkylamines and their acid addition salts and quaternization products. Alkyl here is preferably C₁-C₂₄-alkyl. Preference is given to N,N-diallyl-N-methylamine and N,N-diallyl-N,N-dimethylammonium compounds, such as, for example, the chlorides and bromides.

Suitable monomers d) are also vinyl- and allyl-substituted nitrogen heterocycles, such as N-vinylimidazole and derivatives thereof, vinyl- and allyl-substituted heteroaromatic compounds, such as 2- and 4-vinylpyridine, 2- and 4-allylpyridine, and the salts thereof.

Preferred monomers d) are the N-vinylimidazole derivatives of the general formula (II), in which R¹ to R³ is hydrogen, C₁-C₄-alkyl or phenyl

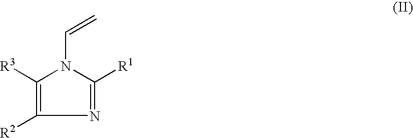

Examples of compounds of the general formula (II) are given in Table 1 below:

TABLE 1

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

Monomer e)

The monomer mixture M) can additionally comprise at least one further monomer e) which is different from the abovementioned monomers a), b) and d) and copolymerizable therewith.

Component e) is preferably used in an amount of from 0 to 50% by weight, particularly preferably 0 to 40% by weight, in particular 0 to 30% by weight, based on the total weight of the components used for the polymerization (i.e. components a), b), c) and, if present d) to g)). If a component e) is used, then it is preferably used in an amount of at least 0.1% by weight, particularly preferably at least 1% by weight, in particular at least 5% by weight.

The additional monomers e) are preferably chosen from N-vinyllactams, open-chain N-vinylamide compounds, esters of α,β-ethylenically unsaturated mono- and dicarboxylic acids with C₁-C₃₀-alkanediols, amides of α,β-ethylenically unsaturated mono- and dicarboxylic acids with C₂-C₃₀-aminoalcohols which have a primary or secondary amino group, primary amides of α,β-ethylenically unsaturated monocarboxylic acids, esters of vinyl alcohol and allyl alcohol with C₁-C₃₀-monocarboxylic acids, vinyl ethers, vinylaromatics, vinyl halides, vinylidene halides, C₁-C₈-monoolefins, nonaromatic hydrocarbons with at least two conjugated double bonds and mixtures thereof.

Suitable additional monomers e) are N-vinyllactams and derivatives thereof which can have, for example, one or more C₁-C₆-alkyl substituents, such as methyl, ethyl, n-propyl, isopropyl, n-butyl, sec-butyl, tert-butyl etc. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc. Preference is given to using N-vinylpyrrolidone and N-vinylcaprolactam.

Open-chain N-vinylamide compounds suitable as monomers e) are, for example, N-vinylformamide, N-vinyl-N-methylformamide, N-vinylacetamide, N-vinyl-N-methylacetamide, N-vinyl-N-ethylacetamide, N-vinylpropionamide, N-vinyl-N-methylpropionamide and N-vinylbutyramide.

Suitable additional monomers e) are also 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate, 3-hydroxy-2-ethylhexyl methacrylate etc.

Suitable additional monomers e) are also acrylamide and methacrylamide.

Suitable additional monomers e) are also vinyl acetate, vinyl propionate, vinyl butyrate and mixtures thereof.

Suitable additional monomers e) are also ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

The abovementioned additional monomers e) can be used individually or in the form of any mixtures.

Crosslinker f)

The copolymers according to the invention can, if desired, comprise at least one copolymerized crosslinker, i.e. a compound with two or more than two ethylenically unsaturated, nonconjugated double bonds.

Crosslinkers are preferably used in an amount of from 0.01 to 3% by weight, particularly preferably 0.1 to 2% by weight, based on the total weight of the components used for the polymerization (i.e. components a), b), c) and, if present, d) to g)).

Suitable crosslinkers f) are, for example, acrylic esters, methacrylic esters, allyl ethers or vinyl ethers of at least dihydric alcohols. The OH groups of the parent alcohols may here be completely or partially etherified or esterified; however, the crosslinkers comprise at least two ethylenically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentylglycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000. Apart from the homopolymers of ethylene oxide and propylene oxide, it is also possible to use block copolymers of ethylene oxide or propylene oxide or copolymers which comprise the ethylene oxide and propylene oxide groups in incorporated form. Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide as the corresponding ethoxylates and propoxylates, respectively. The polyhydric alcohols can also firstly be converted to the corresponding glycidyl ethers by reaction with epichlorohydrin.

Further suitable crosslinkers f) are the vinyl esters or the esters of monohydric unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. However, it is also possible to esterify the monohydric unsaturated alcohols with polybasic carboxylic acids, for example malonic acid, tartaric acid, trimellitic acid, phthalic acid, terephthalic acid, citric acid or succinic acid.

Further suitable crosslinkers f) are esters of unsaturated carboxylic acids with the above described polyhydric alcohols, for example oleic acid, crotonic acid, cinnamic acid or 10-undecenoic acid.

Suitable monomers f) are also straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Further suitable crosslinkers are the acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids, as have been described above.

In addition, triallylamine and triallylmonoalkylammonium salts, e.g. triallylmethylammonium chloride or methylsulfate, are suitable as crosslinker f).

Also suitable are N-vinyl compounds of urea derivatives, at least difunctional amides, cyanurates or urethanes, for example of urea, ethylene urea, propyleneurea or tartaramide, e.g. N,N′-divinylethyleneurea or N,N′-divinylpropyleneurea.

Further suitable crosslinkers are divinyldioxane, tetraallylsilane or tetravinylsilane.

Mixtures of the abovementioned compounds can of course also be used. Preference is given to using water-soluble crosslinkers.

Particularly preferred crosslinkers f) used are, for example, methylenebisacrylamide, triallylamine and triallylalkylammonium salts, divinylimidazole, pentaerythritol triallyl ether, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Very particularly preferred crosslinkers f) are pentaerythritol triallyl ether, methylenebisacrylamide, N,N′-divinylethyleneurea, triallylamine and triallylmonoalkylammonium salts and acrylic esters of glycol, butanediol, trimethylolpropane or glycerol or acrylic esters of glycol, butanediol, trimethylolpropane or glycerol reacted with ethylene oxide and/or epichlorohydrin.

Mono- and Difunctional Regulators g)

In addition to the polyfunctional regulators c) used according to the invention, it is possible to use at least one mono- or difunctional regulator (=component g)) for the preparation of the copolymers. Suitable regulators are described in detail, for example, by K. C. Berger and G. Brandrup in J. Brandrup, E. H. Immergut, Polymer Handbook, 3rd edition, John Wiley & Sons, New York, 1989, pp. II/81-II/141.

The regulators g) are preferably used in a feed amount of from 0 to 10% by weight, particularly preferably from 0 to 8% by weight and in particular from 0 to 5% by weight, based on the total weight of the components used for the polymerization (i.e. components a), b), c) and, if present, d) to g)). If a component g) is used, then it is preferably in an amount of at least 0.01% by weight, particularly preferably at least 0.02% by weight and in particular at least 0.05% by weight. A particularly preferred quantitative range is 0.2 to 1% by weight.

Suitable monofunctional regulators g) are, for example, aldehydes such as formaldehyde, acetaldehyde, propionaldehyde, n-butyraldehyde, isobutyraldehyde.

Additional regulators which may also be used are: formic acid, its salts or esters, such as ammonium formate, 2,5-diphenyl-1-hexene, hydroxylammonium sulfate and hydroxylammonium phosphate.

Further suitable regulators are halogen compounds which have one or two halogen atoms, e.g. dichloromethane, 1,2-dichloroethane, allyl bromide, and benzyl compounds, such as benzyl chloride or benzyl bromide.

Further suitable regulators are allyl compounds, such as, for example, allyl alcohol, functionalized allyl ethers, such as allyl ethoxylates, alkyl allyl ethers, or glycerol monoallyl ether.

The regulators preferably used are compounds which comprise sulfur in bonded form.

Compounds of this type are, for example, inorganic hydrogensulfites, disulfites and dithionites or organic sulfides, disulfides, polysulfides, sulfoxides and sulfones. These include di-n-butyl sulfide, di-n-octyl sulfide, diphenyl sulfide, thiodiglycol, ethylthioethanol, diisopropyl disulfide, di-n-butyl disulfide, di-n-hexyl disulfide, diacetyl disulfide, diethanol sulfide, di-t-butyl trisulfide, dimethyl sulfoxide, dialkyl sulfide, dialkyl disulfide and/or diaryl sulfide.

Particular preference is given to organic compounds which comprise sulfur in bonded form.

Compounds preferably used as polymerization regulators are thiols (compounds which comprise sulfur in the form of SH groups, also referred to as mercaptans). Preferred regulators are mono-, bi- and polyfunctional mercaptans, mercaptoalcohols and/or mercaptocarboxylic acids.

Examples of these compounds are allyl thioglycolates, ethyl thioglycolate, cysteine, 2-mercaptoethanol, 1,3-mercaptopropanol, 3-mercaptopropane-1,2-diol, 1,4-mercaptobutanol, mercaptoacetic acid, 3-mercaptopropionic acid, mercaptosuccinic acid, thioglycerol, thioacetic acid, thiourea and alkyl mercaptans, such as n-butyl mercaptan, n-hexyl mercaptan or n-dodecyl mercaptan.

Particularly preferred thiols are cysteine, 2-mercaptoethanol, 1,3-mercaptopropanol, 3-mercaptopropane-1,2-diol, thioglycerol, thiourea.

Examples of bifunctional regulators which comprise two sulfurs in bonded form are bifunctional thiols, such as, for example dimercaptopropanesulfonic acid (sodium salt), dimercaptosuccinic acid, dimercapto-1-propanol, dimercaptoethane, dimercaptopropane, dimercaptobutane, dimercaptopentane, dimercaptohexane, ethylene glycol bisthioglycolates and butanediol bisthioglycolate.

The invention further provides a method of producing a copolymer with anionogenic and/or anionic groups by free-radical polymerization of a monomer mixture M) comprising
a) at least one compound with a free-radically polymerizable α,β-ethylenically unsaturated double bond and at least one anionogenic and/or anionic group per molecule, and
b) at least one monomer which is chosen from esters of α,β-ethylenically unsaturated mono- and dicarboxylic acids with C₁-C₃₀-alkanols, N-alkyl- and N,N-dialkylamides of α,β-ethylenically unsaturated monocarboxylic acids and mixtures thereof,
   and if appropriate further monomers different from components a) and b) in the presence of
c) at least one compound which has a regulating effect on the molecular weight of the resulting polymers and which has at least three functional regulating groups (polyfunctional regulator).

With regard to the monomers and regulators suitable for producing the copolymers by the method according to the invention reference is made to the above statements regarding components a) to g).

According to the method of the invention, the monomer mixture M) can be polymerized by customary methods known to the person skilled in the art, e.g. by solution, precipitation, suspension or emulsion polymerization. Preference is given to preparation by emulsion or solution polymerization.

The polymerization is generally carried out at temperatures in a range from 0 to 150° C., preferably 20 to 100° C., particularly preferably 30 to 95° C.

The polymerization preferably takes place at atmospheric pressure, although polymerization at increased pressure, for example in a pressure-tight reactor under the autogenous pressure of the components used for the polymerization, is also possible. The suitable pressure range is between 1 and 5 bar.

To produce the copolymers, the monomers can be polymerized with the help of free-radical-forming initiators. Initiators which can be used for the free-radical polymerization are the peroxo and/or azo compounds customary for this purpose, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, tert-butyl peroxy-2-ethylhexanoate, tert-butyl permaleate, cumene hydroperoxide, diisopropyl peroxydicarbamate, bis(o-toloyl)peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, 2,2′-azobis(2-amidinopropane)hydrochloride (V50 from Wako Pure Chemicals Industries, Ltd.), or 2,2′-azobis(2-methylbutyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate, H₂O₂/Cu^{I}.

If the copolymers are prepared by the method of solution polymerization, then the solvent is preferably chosen from water and/or polar organic solvents. Preference is given to aqueous solvents, such as water and mixtures of water with water-miscible solvents, for example alcohols, such as methanol, ethanol, n-propanol, isopropanol, n-butanol, sec-butanol, tert-butanol, n-hexanol and cyclohexanol, and glycols, such as ethylene glycol, propylene glycol and butylene glycol, and the methyl or ethyl ethers of dihydric alcohols, diethylene glycol, triethylene glycol, polyethylene glycols with number-average molecular weights up to about 3000, glycerol and dioxane. Particular preference is given to polymerization in an alcohol or alcohol mixture.

Preparation of the copolymers by the method of free-radical aqueous emulsion polymerization is also possible. Suitable aqueous solvents or dispersants are those specified above for solution polymerization. Preference is given to using water.

The emulsion polymerization can be carried out either as a batch process or in the form of a feed process, including step procedure and gradient procedure. The polymerization preferably takes place as a feed procedure in which some of the polymerization mixture is initially introduced and the other components are added to the initial charge in their entirety or partially, in batches or continuously, together or in separate feeds.

Preferably, at least some of the aqueous medium and if appropriate some of the monomers, regulators and/or interface-active substances used are initially introduced into a polymerization zone, heated to the polymerization temperature, if appropriate this initial charge (if it comprises monomer) is incipiently polymerized, and then the remainder of the polymerization mixture is introduced into the polymerization zone via one or more spatially separate feeds while maintaining the polymerization. Here, polymerization initiator and monomers are usually added in separate feeds.

The introduction of the monomers can take place individually or in the form of mixtures, in pure form or in dissolved form in an aqueous medium or in emulsified form.

The addition of initiator generally takes place via a separate feed, generally in the aqueous phase, although it is possible to combine monomer feed and initiator feed prior to them entering the reaction zone.

The addition of the polyfunctional regulator and if appropriate of further regulators can take place in a mixture with a monomer feed, in a mixture with the initiator feed or separately.

The other components of the monomer emulsion, which are defined more precisely below, are added depending on compatibility together with one of the abovementioned feeds or separately in pure form, as a solution in water or a suitable solvent.

The formation of the emulsion can take place in the reaction zone using suitable mixing devices. Preferably, at least some of the components are already emulsified before being introduced into the reaction zone. Preferably, the mixers are chosen from stirred tanks, rotor-stator systems, preferably colloid mills or tooth rim dispersing machines, ultrasound homogenizers, high-pressure homogenizers, continuous tube mixers, jet dispersers, shear gap mixers, etc.

Suitable interface-active additives are the protective colloids and emulsifiers customarily used as dispersants during emulsion polymerization, as are described, for example, in Houben-Weyl, Methoden der organischen Chemie [Methods of organic chemistry], volume XIV/1, Makromolekulare Stoffe [Molecular substances], Georg-Thieme-Verlag, Stuttgart, 1961, pp. 411 to 420. Suitable additional protective colloids are, for example, polyvinyl alcohols and partially hydrolyzed polyvinyl acetates, polyacrylate, polyvinylpyrrolidone, cellulose and cellulose derivatives, such as, for example, methylcellulose, hydroxyethylcellulose, carboxymethylcellulose, starch and starch derivatives, such as, for example, cyanoalkyl ether starch, hydroxyalkyl ether starch, carboxymethyl starch etc. Suitable emulsifiers are either anionic, cationic or nonionic emulsifiers.

As interface-active substances, preference is given to using emulsifiers whose relative molecular weights are, in contrast to the protective colloids, usually below 3500 daltons.

Nonionic emulsifiers which can be used are araliphatic (alkylated aromatics) or aliphatic nonionic emulsifiers, for example ethoxylated mono-, di- and trialkylphenols (degree of EO: 3 to 50, alkyl radical: C₄-C₁₀), ethoxylates of long-chain alcohols (degree of EO: 3 to 50, alkyl radical: C₈-C₃₆) and polyethylene oxide/polypropylene oxide block copolymers. Preference is given to ethoxylates of long-chain alkanols (alkyl radical C₁₀-C₂₂, average degree of ethoxylation 10 to 50) and of these particular preference is given to those with a linear C₁₂-C₁₈-alkyl radical and an average degree of ethoxylation of from 10 to 50, and also ethoxylated monoalkylphenols.

Suitable anionic emulsifiers are, for example, alkali metal and ammonium salts of alkyl sulfates (alkyl radical: C₈-C₂₂), of sulfuric acid half-esters of ethoxylated alkanols (degree of EO: 2 to 50, alkyl radical: C₁₂-C₁₈) and ethoxylated alkylphenols (degree of EO: 3 to 50, alkyl radical: C₄-C₉), of alkylsulfonic acids (alkyl radical: C₁₂-C₁₈) and of alkylarylsulfonic acids (alkyl radical: C₉-C₁₈). Further suitable emulsifiers are given in Houben-Weyl, Methoden der organischen Chemie [Methods of organic chemistry], volume XIV/1, Makromolekulare Stoffe [Macromolecular substances], Georg-Thieme-Verlag, Stuttgart, 1961, pp. 192-208). Suitable anionic emulsifiers are likewise bis(phenoylsulfonic acid)ether or the alkali metal or ammonium salts thereof which carry a C₄-C₂₄-alkyl group on one or both aromatic rings. These compounds are generally known, e.g. from U.S. Pat. No. 4,269,749, and are commercially available, for example as Dowfax® 2A1 (Dow Chemical Company).

Suitable cationic emulsifiers are preferably quaternary ammonium halides, e.g. trimethylcetylammonium chloride, methyltricetylammonium chloride, benzyltriethylammonium chloride or quaternary compounds of N—C₆-C₂₀-alkylpyridines, -morpholines or -imidazoles, e.g. N-laurylpyridinium chloride.

The amount of emulsifier is generally about 0 to 10% by weight, preferably 0.01 to 5% by weight, based on the amount of monomers to be polymerized.

Polymeric dispersants can additionally be used for the preparation. These additional polymeric dispersants are generally used in amounts of from 0 to 10% by weight, preferably 0.01 to 5% by weight, based on the amount of monomers to be polymerized. The additional monomeric dispersants generally comprise at least one functional group, chosen from carboxyl, carboxylate, ether, hydroxyl, sulfate ester, amino, imino, tert-amino and/or quaternary ammonium groups. Examples of such compounds are: polyacrylic acids, polyvinyl acetate, polyalkylene glycols, in particular polyethylene glycols, polyvinyl alcohol, polyvinylpyridine, polyethyleneimine, polyvinylimidazole, polyvinylsuccinimide and polydiallyldimethylammonium chloride, polyvinylpyrrolidone, polymers which comprise at least 5% by weight of vinylpyrrolidone units, polymers which comprise at least 50% by weight of vinyl alcohol units, oligosaccharides, polysaccharides, oxidatively, hydrolytically or enzymatically degraded polysaccharides, chemically modified oligo- or polysaccharides, such as, for example, carboxymethylcellulose, water-soluble starch and starch derivatives, starch esters, starch xanthanogenates, starch acetates, dextran, and mixtures thereof.

After the polymerization process, the reaction mixtures produced during the polymerization in the form of solutions, dispersions, etc. can be subjected to a physical or chemical after-treatment. Such processes are, for example, the known processes of residual monomer reduction, such as, for example, after-treatment by adding polymerization initiators or mixtures of two or more polymerization initiators at suitable temperatures or heating the polymerization solution to temperatures above the polymerization temperature, an after-treatment of the polymer solution by means of steam or stripping with an inert gas, such as nitrogen, or treatment of the reaction mixture with oxidizing or reducing reagents, adsorption processes, such as the adsorption of contamination on selected media, such as, for example, activated carbon, or an ultrafiltration. It is of course also possible to additionally subject the resulting aqueous polymer dispersion to inert gas and/or steam stripping before or after an after-polymerization step. This stripping process is preferably carried out after the after-polymerization step. As described in EP-A 805 169, a partial neutralization of the dispersion to a pH in the range from 5 to 7, preferably to a pH in the range from 5.5 to 6.5, before the physical deodorization may be advantageous.

The reaction mixtures obtained during the polymerization can be partially or completely neutralized before or after an after-treatment. In particular, if the polymers are used in hair cosmetic preparations, such a partial or complete neutralization is advantageous. Preferably, the anionogenic groups present in the polymers are neutralized in an amount of at least 10%, particularly preferably at least 30%, very particularly preferably at least 40%, in particular at least 50%, specifically at least 70%, more specifically at least 95%. In a particularly preferred embodiment, an essentially complete neutralization is carried out, i.e. to at least 99% and in particular to 100%. For this, it may be advantageous to use the neutralizing agent in a molar excess relative to the groups available for the neutralization.

The bases used for the neutralization may be alkali metal bases, such as sodium hydroxide, potassium hydroxide, sodium carbonate, sodium hydrogencarbonate, potassium carbonate or potassium hydrogencarbonate and alkaline earth metal bases, such as calcium hydroxide, calcium oxide, magnesium hydroxide or magnesium carbonate, and ammonia.

Amines suitable for the neutralization are, for example, C₁-C₁₀-alkylamines, preferably C₅-C₁₀-alkylamines, such as N,N-diethylpropylamine, N,N-dipropylmethylamine and 3-diethylamino-1-propylamine.

Also suitable for the neutralization are mono-, di- and trialkanolamines which preferably have 2 to 5 carbon atoms in the alkanol radicals. Here, the alkanol radicals may also be in etherified form. Preference is given to mono-, di- and triethanolamine, mono-, di- and tri-n-propanolamine, mono-, di- and triisopropanolamine, 2-amino-2-methylpropanol, di(2-methoxyethyl)amine and mixtures thereof.

Also suitable for the neutralization are alkanediolamines which preferably have 2 to 5 carbon atoms in the alkanediol radicals. These include 2-amino-2-methylpropane-1,3-diol and 2-amino-2-ethylpropane-1,3-diol. Particularly good results, particularly when using the polymers in cosmetic compositions are achieved by neutralization with 2-amino-2-methyl-1-propanol, triisopropanolamine, N,N-dimethylaminoethanol or 3-diethylamino-1-propylamine. The neutralization of the acid groups can also be carried out using mixtures of two or more of the abovementioned bases.

Also suitable for the neutralization and for adjusting the pH are aqueous buffer solutions, such as, for example, buffers based on alkali metal and ammonium carbonates or alkali metal and ammonium hydrogencarbonates. The neutralizing agents are then preferably added to the polymer dispersion in the form of a dilute aqueous solution.

The copolymers according to the invention can also be neutralized with further cosmetically or pharmaceutically acceptable polymers.

Polymers suitable for the neutralization are, for example, cationic polymers with the INCI name Polyquaternium, e.g. copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamido copolymers (Polyquaternium-7) and chitosan. Also suitable are vinylimidazole-containing copolymers, such as Luvitev® VPI, Luviset Clear®, copolymers of vinylpyrrolidone, vinylcaprolactam and vinylimidazole, and copolymers of vinylpyrrolidone, methacrylamide, vinylimidazole and N-vinylimidazolium salts. Suitable cationic (quaternized) polymers are also Merquat® (polymer based on dimethyldiallylammonium chloride), Gafquat® (quaternary polymers which are produced by the reaction of polyvinylpyrrolidone with quaternary ammonium compounds), polymer JR (hydroxyethylcellulose with cationic groups) and plant-based cationic polymers, e.g. guar polymers, such as the Jaguar® brands from Rhodia.

In a specific embodiment, silicone polymers with cationogenic groups are used for the neutralization. Suitable silicone polymers are available under the INCI trade name Amodimethicone. These include the products available commercially under the names Wacker-Belsil® ADM 652, ADM 653, ADM 656, ADM 1100, ADM 1600 and ADM 1650. Suitable polymers containing amino groups are also the silicone-aminopolyalkylene oxide block copolymers described in WO 97/32917. Also suitable are the dimethiconebisaminohydroxypropyl copolyols sold by Witco/OSi under the name Silsoft® A-843 and trimethylsilyl-amidomethicone copolymers sold under the name Silsoft® A-858. Also suitable are the silicone-containing polymers described in EP-A-1035144 for the neutralization.

If the polymers according to the invention comprise copolymerized monomers d) and also have free cationogenic groups, then the latter can, as described above, be converted partially or completely into cationic groups by protonation or by quaternization.

The liquid reaction mixtures can be dried and, for example, converted into powder form by various drying methods, such as, for example, spray drying, fluidized spray drying, roller drying or freeze drying. Preference is given to using spray drying. The dried polymer powders obtained in this way can advantageously be converted again into an aqueous solution or dispersion by dissolution or redispersion, respectively, in aqueous media. Pulverulent copolymers have the advantage of better storability, simpler transportability and generally have a lower tendency for microbial attack. Customary preservatives can be used for stabilizing and preserving polymer solutions and dispersions. Preference is given to using hydrogen peroxide.

The invention further relates to the copolymers obtainable by the method according to the invention.

These copolymers are exceptionally suitable for producing cosmetic and pharmaceutical compositions. They serve here, for example, as polymeric film formers in preparations for body care, which involves the application of cosmetic preparations to keratinous surfaces such as skin, hair, nails and also mouth care preparations. They can be used and formulated universally in very diverse cosmetic preparations and are compatible with the customary components. For the same solids content, formulations based on the copolymers according to the invention generally have significantly lower viscosities than corresponding preparations based on polymers known from the prior art, in particular those which have only been prepared in the presence of mono- and/or difunctional regulators. In particular, they permit the preparation of cosmetic and pharmaceutical compositions in the form of sprays, which are characterized by very good spray properties.

As a 5% strength by weight solution in water/ethanol (40/55), the completely neutralized copolymers according to the invention preferably have a viscosity (determined at 25° C. using a rotary viscometer) in the range from 5 to 20 mPas, very particularly preferably from 7 to 14 mPas.

The K values of the copolymers according to the invention are preferably in a range from 20 to 80, particularly preferably from 25 to 40 (measured in accordance with K. Fikentscher, Cellulosechemie [Cellulose chemistry] vol. 13, pp. 58-64 (1932) at 25° C. as a 1% strength solution in ethanol).

The compositions according to the invention have a cosmetically or pharmaceutically acceptable carrier B) which is chosen from
i) water,
ii) water-miscible organic solvents, preferably C₁-C₄-alkanols,
iii) oils, fats, waxes,
iv) esters of C₆-C₃₀-monocarboxylic acids with mono-, di- or trihydric alcohols which are different from iii),
v) saturated acyclic and cyclic hydrocarbons,
vi) fatty acids,
vii) fatty alcohols,
viii) propellant gases
   and mixtures thereof.

The compositions according to the invention have, for example, an oil or fat component B) which is chosen from: hydrocarbons of low polarity, such as mineral oils; linear saturated hydrocarbons, preferably having more than 8 carbon atoms, such as tetradecane, hexadecane, octadecane etc.; cyclic hydrocarbons, such as decahydronaphthalene; branched hydrocarbons; animal and vegetable oils; waxes; wax esters; vaseline; esters, preferably esters of fatty acids, such as, for example, the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkylbenzoates, benzyl benzoate; other cosmetic esters, such as fatty acid triglycerides, propylene glycol monolaurate, polyethylene glycol monolaurate, C₁₀-C₁₅-alkyl lactates, etc. and mixtures thereof.

Suitable silicone oils B) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are commercially available, for example, under the name Cyclomethicone.

Preferred oil and fat components B) are chosen from paraffin and paraffin oils; vaseline; natural fats and oils, such as castor oil, soya oil, peanut oil, olive oil, sunflower oil, sesame oil, avocado oil, cocoa butter, almond oil, peach kernel oil, ricinus oil, cod-liver oil, lard, spermaceti, spermaceti oil, sperm oil, wheat germ oil, macadamia nut oil, evening primrose oil, jojoba oil; fatty alcohols, such as lauryl alcohol, myristyl alcohol, cetyl alcohol, stearyl alcohol, oleyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candelilla wax, spermaceti and mixtures of the abovementioned oil and fat components.

Suitable cosmetically and pharmaceutically compatible oil and fat components B) are described in Karl-Heinz Schrader, Grundlagen und Rezepturen der Kosmetika [Fundamentals and formulations of cosmetics], 2nd edition, Verlag Hüthig, Heidelberg, pp. 319-355, which is hereby incorporated by reference.

Preferred carriers B) are hydrophilic carriers B). Suitable hydrophilic carriers are chosen from water, 1-, 2- or polyhydric alcohols having preferably 1 to 8 carbon atoms, such as ethanol, n-propanol, isopropanol, propylene glycol, glycerol, sorbitol, etc., and mixtures thereof.

Preferred carriers B) are also propellants. Suitable propellants B) are those customarily used for hairsprays or aerosol foams. Preference is given to mixtures of propane/butane, pentane, dimethyl ether, 1,1-difluoroethane (HFC-152 a), carbon dioxide, nitrogen or compressed air.

The cosmetic compositions according to the invention may be a skin cosmetic, hair cosmetic, dermatological, hygiene or pharmaceutical composition. Due to their film-forming properties, the copolymers described above are also especially suitable as additives for hair and skin cosmetics.

Preferably, the compositions according to the invention are in the form of a gel, foam, spray, ointment, cream, emulsion, suspension, lotion, milk or paste. If desired, liposomes or microspheres can also be used. The compositions according to the invention are particularly preferably in the form of a spray.

The compositions according to the invention are specifically a hair-treatment composition in spray form.

The cosmetic or pharmaceutical compositions according to the invention can additionally comprise cosmetically and/or dermatologically active ingredients and auxiliaries.

Preferably, the cosmetic compositions according to the invention comprise at least one copolymer as defined above, at least one carrier B) as defined above and at least one constituent different therefrom which is chosen from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tints, tanning agents, dyes, pigments, consistency regulators, humectants, regreasing agents, collagen, protein hydrolyzates, lipids, antioxidants, antifoams, antistats, emollients and softeners.

Customary thickeners in such formulations are crosslinked polyacrylic acids and derivatives thereof, polysaccharides and derivatives thereof, such as xanthan gum, agar agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone. Preference is given to using nonionic thickeners.

Suitable cosmetically and/or dermatologically active ingredients are, for example, coloring active ingredients, skin and hair pigmentation agents, tints, tanning agents, bleaches, keratin-hardening substances, antimicrobial active ingredients, photofilter active ingredients, repellent active ingredients, substances with hyperemic activity, substances with keratolytic and keratoplastic activity, antidandruff active ingredients, antiphlogistics, substances which have a keratinizing effect, substances which act as antioxidants or as free-radical scavengers, skin moisturizers or humectants, regreasing active ingredients, antierythimatous or antiallergic active ingredients and mixtures thereof.

Artificially skin-tanning active ingredients which are suitable for tanning the skin without natural or artificial irradiation with UV rays are, for example, dihydroxyacetone, alloxan and walnut shell extract. Suitable keratin-hardening substances are usually active ingredients as are also used in antiperspirants, such as, for example, potassium aluminum sulfate, aluminum hydroxychloride, aluminum lactate, etc. Antimicrobial active ingredients are used in order to destroy microorganisms or to inhibit their growth and thus serve both as preservatives and also as a deodorizing substance which reduces the formation or the intensity of body odor. These include, for example, customary preservatives known to the person skilled in the art, such as p-hydroxybenzoates, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine etc. Suitable photofilter active ingredients are substances which absorb UV rays in the UV-B and/or UV-A region. Suitable UV filters are, for example, 2,4,6-triaryl-1,3,5-triazines in which the aryl groups may each carry at least one substituent which is preferably chosen from hydroxyl, alkoxy, specifically methoxy, alkoxycarbonyl, specifically methoxycarbonyl and ethoxycarbonyl and mixtures thereof. Also suitable are p-aminobenzoates, cinnamates, benzophenones, camphor derivatives, and pigments which stop UV rays, such as titanium dioxide, talc and zinc oxide. Suitable repellent active ingredients are compounds which are able to drive away or repel certain animals, in particular insects, from humans. These include, for example, 2-ethyl-1,3-hexanediol, N,N-diethyl-m-toluamide etc. Suitable substances with hyperemic activity, which stimulate blood flow through the skin, are, for example, essential oils, such as dwarf pine, lavender, rosemary, juniper berry, horse chestnut extract, birch leaf extract, hayflower extract, ethyl acetate, camphor, menthol, peppermint oil, rosemary extract, eucalyptus oil, etc. Suitable substances with a keratolytic and keratoplastic effect are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics which counter skin irritations are, for example, allantoin, bisabolol, dragosantol, chamomile extract, panthenol, etc.

The cosmetic compositions according to the invention can comprise, as cosmetic and/or pharmaceutical active ingredient (and also if appropriate as auxiliary), at least one cosmetically or pharmaceutically acceptable polymer which differs from the polymers which form the polyelectrolyte complex used according to the invention. Very generally, these include cationic, amphoteric and neutral polymers.

Suitable polymers are, for example, the cationic polymers described above for the neutralization, which are hereby incorporated by reference.

Further suitable polymers are also neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethyleneimines and salts thereof, polyvinylamines and salts thereof, cellulose derivatives, polyaspartic acid salts and derivatives. These include, for example, Luviflex® Swing (partially saponified copolymer of polyvinyl acetate and polyethylene glycol, BASF).

Suitable polymers are also nonionic, water-soluble or water-dispersible polymers or oligomers, such as polyvinylcaprolactam, e.g. Luviskol® Plus (BASF), or polyvinylpyrrolidone and copolymers thereof, in particular with vinyl esters, such as vinyl acetate, e.g. Luviskol® VA 37 (BASF); polyamides, e.g. based on itaconic acid and aliphatic diamines, as described, for example, in DE-A-43 33 238.

Suitable polymers are also amphoteric or zwitterionic polymers, such as the octylacrylamide/methyl methacrylate/tert-butylaminoethyl methacrylate/2-hydroxypropyl methacrylate copolymers obtainable under the names Amphomer® (National Starch), and zwitterionic polymers, as are disclosed, for example, in the German patent applications DE 39 29 973, DE 21 50 557, DE 28 17 369 and DE 37 08 451. Acrylamidopropyltrimethylammonium chloride/acrylic acid or methacrylic acid copolymers and the alkali metal and ammonium salts thereof are preferred zwitterionic polymers. Further suitable zwitterionic polymers are methacroylethylbetaine/methacrylate copolymers, which are available commercially under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or -dispersible polymers, e.g. polyether siloxanes, such as Tegopren® (Goldschmidt) or Belsil® (Wacker).

The formulation base of pharmaceutical compositions according to the invention preferably comprises pharmaceutically acceptable auxiliaries. Pharmaceutically acceptable auxiliaries are the auxiliaries which are known for use in the fields of pharmacy, food technology and related fields, in particular the auxiliaries listed in the relevant pharmacopeias (e.g. DAB Ph. Eur. BP NF), and other auxiliaries whose properties do not preclude a physiological application.

Suitable auxiliaries may be: glidants, wetting agents, emulsifying and suspending agents, preservatives, antioxidants, antiirritative substances, chelating agents, emulsion stabilizers, film formers, gel formers, odor-masking agents, resins, hydrocolloids, solvents, solubility promoters, neutralizing agents, permeation accelerators, pigments, quaternary ammonium compounds, regreasing and supergreasing agents, ointment bases, cream bases or oil bases, silicone derivatives, stabilizers, sterilizing agents, propellants, drying agents, opacifiers, thickeners, waxes, softeners, white oils. Formulation in this regard is based on expert knowledge, as given, for example, in Fiedler, H. P. Lexikon der Hilfsstoffe für Pharmazie, Kosmetik und angrenzende Gebiete [Lexicon of auxiliaries for pharmacy, cosmetics and related fields], 4^{th} edition, Aulendorf: ECV-Editio-Cantor-Verlag, 1996.

To prepare the dermatological compositions according to the invention the active ingredients can be mixed or diluted with a suitable auxiliary (excipient). Excipients may be solid, semisolid or liquid materials which can serve as a vehicle, carrier or medium for the active ingredient. The admixing of further auxiliaries takes place if desired in the manner known to the person skilled in the art. In addition, the polymers P) and dispersions Pd) are suitable as auxiliaries in pharmacy, preferably as or in (a) coating(s) or binder(s) for solid medicaments. They can also be used in creams and as tablet coatings and tablet binders.

According to a suitable embodiment, the compositions according to the invention are a skin-cleansing composition.

Preferred skin-cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, shower and bath preparations, such as washing lotions, shower baths and shower gels, foam baths, oil baths and scrub preparations, shaving foams, shaving lotions and shaving creams.

According to a further suitable embodiment, the compositions according to the invention are cosmetic compositions for the care and protection of the skin, nail care compositions or preparations for decorative cosmetics.

Suitable skin cosmetic compositions are, for example, face tonics, face masks, deodorants and other cosmetic lotions. Compositions for use in decorative cosmetics comprise, for example, concealing sticks, stage make-up, mascara and eyeshadows, lipsticks, kohl pencils, eyeliners, blushers, powders and eyebrow pencils.

Furthermore, the copolymers according to the invention can be used in nose strips for pore cleansing, in antiacne compositions, repellents, shaving compositions, depilatories, intimate care compositions, footcare compositions and in babycare.

The skincare compositions according to the invention are, in particular, W/O or O/W skin creams, day and night creams, eye creams, face creams, antiwrinkle creams, moisturizing creams, bleaching creams, vitamin creams, skin lotions, care lotions and moisturizing lotions.

Skin cosmetic and dermatological compositions based on the above-described copolymers exhibit advantageous effects. The polymers can, inter alia, contribute to the moisturizing and conditioning of the skin and to an improvement in the skin sensation. The polymers can also act as thickeners in the formulations. By adding the polymers according to the invention, it is possible to achieve a considerable improvement in skin compatibility in certain formulations.

Skin cosmetic and dermatological compositions preferably comprise at least one copolymer according to the invention in an amount of from about 0.001 to 30% by weight, preferably 0.01 to 20% by weight, very particularly preferably 0.1 to 12% by weight, based on the total weight of the composition.

Depending on the field of use, the compositions according to the invention can be applied in a form suitable for skincare, such as, for example, as cream, foam, gel, stick, mousse, milk, spray (pump spray or spray containing propellant) or lotion.

Besides the copolymers according to the invention and suitable carriers, the skin cosmetic preparations can also comprise further active ingredients and auxiliaries customary in skin cosmetics, as described above. These include, preferably, emulsifiers, preservatives, perfume oils, cosmetic active ingredients, such as phytantriol, vitamin A, E and C, retinol, bisabolol, panthenol, photoprotective agents, bleaches, colorants, tinting agents, tanning agents, collagen, protein hydrolyzates, stabilizers, pH regulators, dyes, salts, thickeners, gel formers, consistency regulators, silicones, moisturizers, regreasing agents and further customary additives.

Preferred oil and fat components of the skin cosmetic and dermatological compositions are the abovementioned mineral and synthetic oils, such as, for example, paraffins, silicone oils and aliphatic hydrocarbons with more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, vaseline, hydrogenated lanolin and acetylated lanolin, and mixtures thereof.

The polymers according to the invention can also be mixed with conventional polymers where specific properties are to be set.

To set certain properties, such as, for example, improving the feel to the touch, the spreading behavior, the water resistance and/or the binding of active ingredients and auxiliaries, such as pigments, the skin cosmetic and dermatological preparations can also additionally comprise conditioning substances based on silicone compounds. Suitable silicone compounds are, for example, polyalkylsiloxanes, polyarylsiloxanes, polyarylalkylsiloxanes, polyether siloxanes or silicone resins.

The cosmetic or dermatological preparations are prepared by customary methods known to the person skilled in the art.

The cosmetic and dermatological compositions are preferably in the form of emulsions, in particular water-in-oil (W/O) emulsions or oil-in-water (O/W) emulsions. It is, however, also possible to choose other types of formulation, for example hydro-dispersions, gels, oils, oleogels, multiple emulsions, for example in the form of W/O/W or O/W/O emulsions, anhydrous ointments or ointment bases, etc.

The emulsions are prepared by known methods. Besides at least one copolymer according to the invention, the emulsions usually comprise customary constituents, such as fatty alcohols, fatty acid esters and, in particular, fatty acid triglycerides, fatty acids, lanolin and derivatives thereof, natural or synthetic oils or waxes and emulsifiers in the presence of water. The choice of emulsion type-specific additives and the preparation of suitable emulsions is described, for example, in Schrader, Grundlagen und Rezepturen der Kosmetika [Fundamentals and formulations of cosmetics], Hüthig Buch Verlag, Heidelberg, 2^{nd} edition, 1989, third part, which is hereby expressly incorporated by reference.

A suitable emulsion, e.g. for a skin cream etc., generally comprises an aqueous phase which is emulsified by means of a suitable emulsifier system in an oil or fatty phase. To provide the aqueous phase, a copolymer according to the invention can be used.

Preferred fatty components which the fatty phase of the emulsions may comprise are: hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karité oil, hoplostethus oil; mineral oils whose distillation start-point under atmospheric pressure is about 250° C. and whose distillation end-point is 410° C., such as, for example, Vaseline oil; esters of saturated or unsaturated fatty acids, such as alkyl myristates, e.g. i-propyl, butyl or cetyl myristate, hexadecyl stearate, ethyl or isopropyl palmitate, octanoic or decanoic acid triglycerides and cetyl ricinoleate.

The fatty phase may also comprise silicone oils soluble in other oils, such as dimethylpolysiloxane, methylphenylpolysiloxane and the silicone glycol copolymer, fatty acids and fatty alcohols.

Besides the copolymers according to the invention, it is also possible to use waxes, such as, for example, carnauba wax, candelilla wax, beeswax, microcrystalline wax, ozokerite wax and the oleates, myristates, linoleates and stearates of Ca, Mg and Al.

In addition, an emulsion according to the invention may be in the form of an O/W emulsion. Such an emulsion usually comprises an oil phase, emulsifiers which stabilize the oil phase in the water phase, and an aqueous phase which is usually present in thickened form. Suitable emulsifiers are preferably O/W emulsifiers, such as polyglycerol esters, sorbitan esters or partially esterified glycerides.

According to a further preferred embodiment, the compositions according to the invention are a shower gel, a shampoo formulation or a bath preparation.

Such formulations comprise at least one polyelectrolyte complex A) and customarily anionic surfactants as base surfactants and amphoteric and/or nonionic surfactants as cosurfactants. Other suitable active ingredients and/or auxiliaries are generally chosen from lipids, perfume oils, dyes, organic acids, preservatives and antioxidants, and thickeners/gel formers, skin conditioning agents and humectants.

These formulations preferably comprise 2 to 50% by weight, preferably 5 to 40% by weight, particularly preferably 8 to 30% by weight, of surfactants, based on the total weight of the formulation.

All anionic, neutral, amphoteric or cationic surfactants customarily used in body-cleansing compositions can be used in the washing, shower and bath preparations.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 moles per mol of alcohol. Also suitable are alkylamine oxides, mono- or dialkylalkanolamides, fatty acid esters of polyethylene glycols, ethoxylated fatty acid amides, alkyl polyglycosides or sorbitan ether esters.

Furthermore, the washing, shower and bath preparations can also comprise customary cationic surfactants such as, for example, quaternary ammonium compounds, for example cetyltrimethylammonium chloride.

In addition, the shower gel/shampoo formulations can comprise thickeners, such as, for example, sodium chloride, PEG-55, propylene glycol oleate, PEG-120 methylglucose dioleate and others, and also preservatives, further active ingredients and auxiliaries and water.

According to a further preferred embodiment, the compositions according to the invention are hair-treatment compositions.

Hair-treatment compositions according to the invention preferably comprise at least one copolymer according to the invention in an amount in the range from about 0.1 to 30% by weight, preferably 0.5 to 20% by weight, based on the total weight of the composition.

The hair-treatment compositions according to the invention are preferably in the form of a setting foam, hair mousse, hair gel, shampoo, hairspray, hair foam, end fluid, neutralizer for permanent waves, hair colorant and bleach or “hot-oil treatment”. Depending on the field of use, the hair cosmetic preparations can be applied in the form of an (aerosol) spray, (aerosol) foam, gel, gel spray, cream, lotion or wax. Hairsprays comprise here both aerosol sprays and also pump sprays without propellant gas. Hair foams comprise both aerosol foams and also pump foams without propellant gas. Hairsprays and hair foams preferably comprise predominantly or exclusively water-soluble or water-dispersible components. If the compounds used in the hairsprays and hair foams according to the invention are water-dispersible, they can be used in the form of aqueous microdispersions with particle diameters of customarily 1 to 350 nm, preferably 1 to 250 nm. The solids contents of these preparations are usually in a range from about 0.5 to 20% by weight. These microdispersions generally require no emulsifiers or surfactants for their stabilization.

In a preferred embodiment, the hair cosmetic formulations according to the invention comprise
a) 0.05 to 20% by weight of at least one copolymer A) according to the invention,
b) 20 to 99.95% by weight of water and/or alcohol,
c) 0 to 50% by weight of at least one propellant gas,
d) 0 to 5% by weight of at least one emulsifier,
e) 0 to 3% by weight of at least one thickener, and
f) up to 25% by weight of further constituents.

Alcohol is understood as meaning all alcohols customary in cosmetics, e.g. ethanol, isopropanol, n-propanol.

Further constituents are understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein hydrolyzates, alpha- and beta-hydroxycarboxylic acids, stabilizers, pH regulators, dyes, viscosity regulators, gel formers, salts, moisturizers, regreasing agents, complexing agents and further customary additives.

Also included here are all styling and conditioner polymers known in cosmetics which can be used in combination with the polymers according to the invention if quite specific properties are to be set.

Suitable conventional hair cosmetic polymers are, for example, the abovementioned cationic, anionic, neutral, nonionic and amphoteric polymers, which are hereby incorporated by reference.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyether siloxanes, silicone resins or dimethicone copolyols (CTFA) and aminofunctional silicone compounds, such as amidomethicone (CTFA).

The copolymers A) according to the invention can be used in cosmetic preparations as setting and/or conditioning agents. They are particularly suitable as setting agents in hairstyling preparations, in particular hairsprays (aerosol sprays and pump sprays without propellant gas) and hair foams (aerosol foams and pump foams without propellant gas).

In a preferred embodiment, the compositions according to the invention are in the form of a spray. They then preferably comprise a device for generating an aerosol, comprising
a pressurized container comprising at least one copolymer A), as defined above, a liquid solvent, if appropriate a propellant and optionally further ingredients, and
a spray device.

If the compositions according to the invention are formulated in the form of a spray, e.g. in the form of a hairspray, they comprise, in a first embodiment, a sufficient amount of a propellant. Suitable propellants are fluorinated hydrocarbons, such as 1,1-difluoroethane (HFC-152 a). Suitable propellants are preferably hydrocarbons (LPG), in particular propane, n-butane, n-pentane and mixtures thereof. Suitable hydrocarbon mixtures are propane/butane mixtures. A suitable alternative to the hydrocarbon propellants is, in particular, dimethyl ether. Further preferred propellants are compressed gases, such as nitrogen, air or carbon dioxide. The above-described copolymers A) used in the compositions according to the invention have a high propellant gas compatibility, in particular a high compatibility to dimethyl ether, and can be formulated to give products with a high propellant gas content of, for example, at least 40% by weight, preferably at least 50% by weight, based on the total weight of the composition. In general, however, it is also possible to keep the propellant content low in order to formulate products with a low VOC content. In such products, the propellant gas content is then generally not more than 55% by weight, based on the total weight of the composition.

According to a second embodiment, the compositions according to the invention are also suitable for preparations without the addition of propellants. If the compositions according to the invention are formulated without the addition of propellants, the aerosol device additionally comprises a device for generating pressure, e.g. a pump device.

In a preferred embodiment, spray preparations comprise
a) 0.1 to 10% by weight of at least one copolymer A),
b) 20 to 99.9% by weight of water and/or alcohol,
c) 0 to 70% by weight of at least one propellant,
d) 0 to 20% by weight of further constituents.

A formulation for aerosol foams preferred according to the invention comprises
a) 0.1 to 10% by weight of at least one copolymer A),
b) 55 to 99.8% by weight of water and/or alcohol,
c) 5 to 20% by weight of a propellant,
d) 0.1 to 5% by weight of an emulsifier,
e) 0 to 10% by weight of further constituents.

The emulsifiers used may be all emulsifiers customarily used in hair foams. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ethers; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, quaternium-1 to x (INCI).

Anionic emulsifiers may be chosen, for example, from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

The copolymers A) according to the invention can also be used in shampoo formulations. Preferred shampoo formulations comprise
a) 0.05 to 10% by weight of at least one copolymer A),
b) 25 to 94.95% by weight of water,
c) 5 to 50% by weight of surfactants,
c) 0 to 5% by weight of a further conditioning agent,
d) 0 to 10% by weight of further cosmetic constituents.

In the shampoo formulations it is possible to use all anionic, neutral, amphoteric or cationic surfactants customarily used in shampoos.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

For example, sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate are suitable.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates. For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 moles per mole of alcohol. In addition, alkylamine oxides, mono- or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethylammonium chloride.

In the shampoo formulations, in order to achieve certain effects, customary conditioners can be used in combination with the polyelectrolyte complexes A). These include, for example, the abovementioned cationic polymers with the INCI name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamide copolymers (Polyquaternium-7). It is also possible to use protein hydrolyzates, and conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and aminofunctional silicone compounds, such as amidomethicone (CTFA). It is also possible to use cationic guar derivatives, such as Guar Hydroxypropyltrimonium Chloride (INCI).

The invention further provides the use of a polyelectrolyte complex, as defined above, as auxiliary in pharmacy, preferably as or in (a) coating(s) for solid drug forms, for modifying rheological properties, as surface-active compound, as or in (an) adhesive(s), and as or in (a) coating(s) for the textile, paper, printing and leather industry.

The invention is explained in more detail by reference to the following nonlimiting examples.

### EXAMPLES

I. Preparation of Copolymers (Emulsion Polymerization)

### Comparative Example C1

### n-Dodecyl Mercaptan as Regulator

By mixing the following components in the given order a monomer emulsion is prepared:
170 g of deionized water
8 g of a 15% strength by weight solution of sodium lauryl sulfate in deionized water
9 g of Tween® 80 (poly(oxyethylene-20)sorbitan monooleate)
264 g of t-butyl acrylate
35 g of ethyl acrylate
49 g of methacrylic acid
2.2 g of n-dodecyl mercaptan

At 25° C., 400 g of deionized water, 1 g of a 15% strength by weight solution of sodium lauryl sulfate in deionized water and 40 g of the finished monomer emulsion are mixed in a reaction vessel. 10 g of a 7% strength by weight solution of sodium persulfate in deionized water are then added and the initial charge is heated to 85° C. After reaching 85° C., the remaining amount of the monomer emulsion is added in the feed process uniformly over a period of 2.5 hours, during which the reaction temperature is kept at 85° C. When the addition is complete, the mixture is after-polymerized for a further 2 hours at 85° C. The reaction mixture is then cooled to 60° C. and 2 g of a 30% strength aqueous hydrogen peroxide solution are added. The polymerization mixture is then adjusted to a pH of 6.5 using a 10% strength by weight solution of ammonium hydrogencarbonate in deionized water. Steam distillation is then carried out for 30 minutes.

### Example 2 (According to the Invention)

### 1,1,1-tris(hydroxymethyl)propane tris(2-mercaptopropionate) as Regulator

By mixing the following components in the given order a monomer emulsion is prepared:
170 g of deionized water
8 g of a 15% strength by weight solution of sodium lauryl sulfate in deionized water
9 g of Tween 80
264 g of t-butyl acrylate
35 g of ethyl acrylate
49 g of methacrylic acid
2.2 g of trimethylolpropane tri(mercaptopropionate)

In a reaction vessel, at 25° C., 400 g of deionized water, 1 g of a 15% strength by weight solution of sodium lauryl sulfate in deionized water and 40 g of the finished monomer emulsion are mixed. 10 g of a 7% strength by weight solution of sodium persulfate in deionized water are then added and the initial charge is heated to 85° C. After reaching the polymerization temperature, the remaining amount of the monomer emulsion is added in the feed process uniformly over a period of 2.5 hours, during which the reaction temperature is kept at 85° C. When the addition is complete, the mixture is after-polymerized for a further 2 hours at 85° C. The reaction mixture is then cooled to 60° C. and 2 g of an aqueous 30% strength hydrogen peroxide solution are added. The polymerization mixture is then adjusted to a pH of 6.5 with a 10% strength by weight solution of ammonium hydrogencarbonate in deionized water. Steam distillation is then carried out for 30 minutes.

The polymers in Table 1 below were synthesized according to the general experimental procedures specified above.

TABLE 1

| | | Weight ratio | | |
|---|---|---|---|---|
| | | of the | | Mol % of |
| Example | Monomers | monomers | Regulator | regulator* |
| C1 | t-BA/EA/MAA | 76/10/14 | n-DMK | 0.36 |
| 2 | t-BA/EA/MAA | 76/10/14 | TMPTMP | 0.36 |
| C3 | t-BA/EA/MAA | 67/10/23 | n-DMK | 0.24 |
| 4 | t-BA/EA/MAA | 67/10/23 | TMPTMP | 0.24 |
| C5 | t-BA/EA/MAA | 73/2/25 | n-DMK | 0.3 |
| 6 | t-BA/EA/MAA | 73/2/25 | TMPTMP | 0.3 |
| 7 | MMA/n-BA/MAA | 65/10/25 | TMPTMA | 0.4 |
| 8 | MMA/n-BA/MAA/AA | 65/10/2/5 | TMPTMP | 0.4 |
| 9 | EMA/MAA | 75/25 | TMPTMP | 0.4 |
| 10 | t-BA/HEMA/MAA | 65/15/20 | TMPTMP | 0.4 |
| C11 | t-BA/EA/MAA | 67/10/23 | n-DMK | 0.52 |

*based on monomers (mol)
t-BA tert-butyl acrylate
AA acrylic acid
MAA methacrylic acid
EA ethyl acrylate
MMA methyl methacrylate
EMA ethyl methacrylate
n-DMK n-dodecyl mercaptan
TMPTMA 1,1,1-tris(hydroxymethyl)propane tris(2-mercaptoacetate)
TMPTMP 1,1,1-tris(hydroxymethyl)propane tris(3-mercaptopropionate)

II. Application Properties

The application properties are given in Table 2 below

TABLE 2

| | VOC 55, 5% SC | Clarity | Flexural | Spray | Stiffening |
|---|---|---|---|---|---|
| Ex.- | Dyn. viscosity | as | rigidity | pattern | effect on |
| No. | [mPas] | aerosol | [cN] | [μm] | the hair |
| C1 | 9.5 | slightly | 206 | 85 | good |
| | | cloudy | | | |
| 2 | 7.8 | clear | 202 | 34 | good |
| C3 | 18 | clear | 340 | not | very |
| | | | | sprayable | good |
| 4 | 13 | clear | 240 | 68 | good |
| C5 | 14 | clear | 230 | 65 | good |
| C11 | 10 | clear | 175 | 55 | weak |

Polymers which are prepared using multifunctional regulators according to the invention are advantageously suitable as setting polymers for formulations with a high water content (low VOC). They are characterized by good setting and a good spray pattern.

Spray Pattern:

Determination of the Particle Size Distribution—Spray Pattern

The particle sizes of the liquid aerosols were determined using the method of scattered light analysis with a commercial Malvern™ Master Sizer X (Malvern Instruments Inc., Southborough Mass., USA).

Measuring Principle:

The measuring system is based on the diffraction of laser light at the particle. Apart from being suitable for the spray analysis (aerosols, pump sprays), this method is also suitable for determining the size of solids, suspensions and emulsions in the size range from 0.1 μm to 2000 μm.

A particle collective (=droplet) is illuminated by a laser. At each droplet, some of the incident laser light is scattered. This light is received on a multielement detector and the associated light energy distribution is determined. From this data, the associated particle distribution is calculated using the evaluation software.

Procedure:

The aerosols were sprayed in at a distance of 29.5 cm to the laser beam. The spray cone entered at a right angle to the laser beam.

Before each measurement, the aerosol cans were fixed to a firmly installed holding device to ensure that all of the aerosols to be tested were measured at exactly the same distance.

Before the actual particle measurement, a background measurement was carried out. In principle, this allows the effects of dust and other contaminants in the test area to be eliminated from the measurements.

The aerosol was then sprayed into the test space. The total particle volume was ascertained over a test period of 2 seconds and evaluated.

Evaluation:

The evaluation comprised a tabular representation over 32 class widths from 0.5 μm to 2000 μm and additionally a graphical representation of the particle size distribution.

Since the spray experiments are an approximately uniform distribution, the mean diameter D(v,0.5) was given.

For readily sprayable aerosol systems in the cosmetics sector this value is below 120 μm, preferably below 100 μm, particularly preferably in the range from 30 μm to 70 μm, depending on polymer content, valve, spray head geometry, solvent ratio and amounts of propellant gas.

The following were used:
as valve A: Seaquist Perfect; cone 0.32 mm, 0.50 VPH 0.40 mm (239436)
as spray head: SK1 (yellow); DU381

Determination of the Flexural Rigidity

The setting of polymeric film formers is measured not only by subjective assessment, but also physically as the flexural rigidity of thin tresses of hair which have been treated with the polymer solution and dried again. In this method, a force transducer determines the force required for the bending, while the entire measurement is carried out under standardized conditions in a conditioned room at 65% relative atmospheric humidity.

To measure the flexural rigidity, 3.0% strength by weight solutions of the polymers according to the invention were prepared. The flexural rigidity was measured on 5 to 10 hair tresses (each about 3 g and 24 cm in length) at 20° C. and 65% relative humidity. The weighed dry hair tresses were immersed into the 3.0% strength by weight polymer solution, with triple immersion and removal ensuring uniform distribution. The excess film former solution was then stripped off between thumb and forefinger and the hair tresses were then carefully squeezed by pressing between filter paper. The tresses were then shaped by hand so that they had a round cross section.

Drying was carried out overnight at 20° C. and 65% relative humidity in the conditioned room.

The tests were carried out in a conditioned room at 20° C. and 65% relative humidity using a tensile/pressure testing instrument. The hair tress was placed symmetrically on two cylindrical rolls of the sample holder. The tress was then bent 40 mm exactly in the middle from above using a rounded punch (breakage of the polymer film). The force required for this was measured using a load cell (50 N) and given in Newtons.

Stiffening Effect on the Hair

To determine the stiffening effect on the hair, 3.0% strength by weight solutions of the polymers according to the invention were prepared. The determination of the stiffening effect on the hair was carried out on 3 hair tresses (each about 3 g and 24 cm in length) at 20° C. and 65% relative humidity. The weighed dry hair tresses were immersed into the 3.0% strength by weight polymer solution, with triple immersion and removal ensuring uniform distribution. The excess film former solution was then stripped off between thumb and forefinger and the hair tresses were then carefully squeezed by pressing between filter paper. The tresses were then shaped by hand such that they had a round cross section.

Drying was carried out overnight at 20° C. and 65% relative humidity in the conditioned room. The stiffening effect on the hair was assessed subjectively by trained personnel.

Viscosity

The viscosity of the polymers in an ethanol/water mixture of the following composition was assessed:
5 g of the copolymer (solid), 100% neutralized with aminomethylpropanol
55 g of ethanol
40 g of water

The viscosity of the solution at 25° C. was then determined using a rotary viscometer from Haake (Rotovisco RV 20) with a measurement device NV (low viscosity) at 500 rpm.

Preferably, the polymers have a viscosity between 5 and 20 mPas, very particularly preferably a viscosity between 7 and 14 mPas.

Propellant Gas Compatibility

To determine the propellant gas compatibility, the following aerosol formulation was prepared in pressurized gas containers made from glass.
5 g of the copolymer (solid), 100% neutralized with aminomethylpropanol
40 g of dimethyl ether
15 g of ethanol
40 g of water

The propellant gas compatibility was assessed visually.

1. A cosmetic or pharmaceutical composition comprising:
A) at least one copolymer with anionogenic and/or anionic groups obtainable by free-radical polymerization of a monomer mixture M) comprising:
a) at least one compound with a free-radically polymerizable α,β-ethylenically unsaturated double bond and at least one anionogenic and/or anionic group per molecule, and
b) at least one monomer which is chosen from esters of α,β-ethylenically unsaturated mono- and dicarboxylic acids with C₁-C₃₀-alkanols, N-alkyl- and N,N-dialkylamides, α,β-ethylenically unsaturated monocarboxylic acids, and mixtures thereof,
in the presence of
c) at least one compound which has a regulating effect on the molecular weight of the resulting polymers and which has at least 3 functional regulating groups (polyfunctional regulator); and
B) at least one cosmetically or pharmaceutically acceptable carrier.

2. The composition according to claim 1, wherein component a) is chosen from monoethylenically unsaturated carboxylic acids, sulfonic acids, phosphonic acids and mixtures thereof.

3. The composition according to claim 1, wherein component c) is chosen from trifunctional and tetrafunctional mercaptans.

4. The composition according to claim 1, wherein the monomer mixture M) additionally comprises at least one compound d) with a free-radically polymerizable α,β-ethylenically unsaturated double bond and a cationogenic and/or cationic group per molecule, with the proviso that the mole fraction of cationogenic and cationic groups of component d) is less than the mole fraction of anionogenic and ionic groups of component a).

5. The composition according to claim 1, wherein the monomer mixture M) additionally comprises at least one further monomer e) which is chosen from N-vinyllactams, open-chain N-vinylamide compounds, esters of α,β-ethylenically unsaturated mono- and dicarboxylic acids with C₁-C₃₀-alkanediols, amides of α,β-ethylenically unsaturated mono- and dicarboxylic acids with C₂-C₃₀-aminoalcohols which have a primary or secondary amino group, primary amides of α,β-ethylenically unsaturated monocarboxylic acids, esters of vinyl alcohol and allyl alcohol with C₁-C₃₀-monocarboxylic acids, vinyl ethers, vinylaromatics, vinyl halides, vinylidene halides, C₁-C₈-monoolefins, nonaromatic hydrocarbons with at least two conjugated double bonds and mixtures thereof.

6. The composition according to claim 1, wherein the monomer mixture M) additionally comprises at least one free-radically polymerizable crosslinking compound f) with at least two α,β-ethylenically unsaturated double bonds per molecule.

7. The composition according to claim 1, wherein the polymerization additionally takes place in the presence of at least one compound g) which has a regulating effect on the molecular weight of the resulting polymers and which has one or two functional regulating groups.

8. The composition according to claim 1, wherein component B) is chosen from
i) water,
ii) water-miscible organic solvents, preferably C₁-C₄-alkanols,
iii) oils, fats, waxes,
iv) esters of C₆-C₃₀-monocarboxylic acids with mono-, di- or trihydric alcohols different from iii),
v) saturated acyclic and cyclic hydrocarbons,
vi) fatty acids,
vii) fatty alcohols,
viii) propellants
and mixtures thereof.

9. The composition according to claim 1, further comprising at least one constituent different from component A) and chosen from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tinting agents, tanning agents, dyes, pigments, consistency regulators, humectants, refatting agents, collagen, protein hydrolysates, lipids, antioxidants, antifoams, antistats, emollients and softeners.

10. The composition according to claim 1, wherein the composition is in the form of a spray.

11. The composition according to claim 10, comprising a device for generating an aerosol, comprising:
a pressurized container comprising at least one copolymer A), a liquid solvent, if appropriate a propellant and if appropriate further ingredients, and
a spray device.

12. A method of producing a copolymer A) with anionogenic and/or anionic groups by free-radical polymerization of a monomer mixture M) comprising:
a) at least one compound with a free-radically polymerizable α,β-ethylenically unsaturated double bond and at least one anionogenic and/or anionic group per molecule; and
b) at least one monomer which is chosen from esters of α,β-ethylenically unsaturated mono- and dicarboxylic acids with C₁-C₃₀-alkanols, N-alkyl- and N,N-dialkylamides of α,β-ethylenically unsaturated monocarboxylic acids and mixtures thereof,
and if appropriate further monomers different from components a) and b) in the presence of
c) at least one compound which has a regulating effect on the molecular weight of the resulting polymers and which has at least three functional regulating groups (polyfunctional regulator).

13. A copolymer A) obtainable by a method as defined in claim 12.

14. (canceled)

15. The composition according to claim 2, wherein component c) is selected from the group consisting of trifunctional and tetrafunctional mercaptans.

16. The composition according to claim 2, wherein the monomer mixture M) additionally comprises at least one compound d) with a free-radically polymerizable α,β-ethylenically unsaturated double bond and a cationogenic and/or cationic group per molecule, with the proviso that the mole fraction of cationogenic and cationic groups of component d) is less than the mole fraction of anionogenic and ionic groups of component a).

17. The composition according to claim 3, wherein the monomer mixture M) additionally comprises at least one compound d) with a free-radically polymerizable α,β-ethylenically unsaturated double bond and a cationogenic and/or cationic group per molecule, with the proviso that the mole fraction of cationogenic and cationic groups of component d) is less than the mole fraction of anionogenic and ionic groups of component a).

18. The composition according to claim 2, wherein the monomer mixture M) additionally comprises at least one further monomer e) which is chosen from N-vinyllactams, open-chain N-vinylamide compounds, esters of α,β-ethylenically unsaturated mono- and dicarboxylic acids with C₁-C₃₀-alkanediols, amides of α,β-ethylenically unsaturated mono- and dicarboxylic acids with C₂-C₃₀-aminoalcohols which have a primary or secondary amino group, primary amides of α,β-ethylenically unsaturated monocarboxylic acids, esters of vinyl alcohol and allyl alcohol with C₁-C₃₀-monocarboxylic acids, vinyl ethers, vinylaromatics, vinyl halides, vinylidene halides, C₁-C₈-monoolefins, nonaromatic hydrocarbons with at least two conjugated double bonds and mixtures thereof.

19. The composition according to claim 3, wherein the monomer mixture M) additionally comprises at least one further monomer e) which is chosen from N-vinyllactams, open-chain N-vinylamide compounds, esters of α,β-ethylenically unsaturated mono- and dicarboxylic acids with C₁-C₃₀-alkanediols, amides of α,β-ethylenically unsaturated mono- and dicarboxylic acids with C₂-C₃₀-aminoalcohols which have a primary or secondary amino group, primary amides of α,β-ethylenically unsaturated monocarboxylic acids, esters of vinyl alcohol and allyl alcohol with C₁-C₃₀-monocarboxylic acids, vinyl ethers, vinylaromatics, vinyl halides, vinylidene halides, C₁-C₈-monoolefins, nonaromatic hydrocarbons with at least two conjugated double bonds and mixtures thereof.

20. The composition according to claim 3, wherein the monomer mixture M) additionally comprises at least one further monomer e) which is chosen from N-vinyllactams, open-chain N-vinylamide compounds, esters of α,β-ethylenically unsaturated mono- and dicarboxylic acids with C₁-C₃₀-alkanediols, amides of α,β-ethylenically unsaturated mono- and dicarboxylic acids with C₂-C₃₀-aminoalcohols which have a primary or secondary amino group, primary amides of α,β-ethylenically unsaturated monocarboxylic acids, esters of vinyl alcohol and allyl alcohol with C₁-C₃₀-monocarboxylic acids, vinyl ethers, vinylaromatics, vinyl halides, vinylidene halides, C₁-C₈-monoolefins, nonaromatic hydrocarbons with at least two conjugated double bonds and mixtures thereof.

21. The composition according to claim 4, wherein the monomer mixture M) additionally comprises at least one further monomer e) which is chosen from N-vinyllactams, open-chain N-vinylamide compounds, esters of α,β-ethylenically unsaturated mono- and dicarboxylic acids with C₁-C₃₀-alkanediols, amides of α,β-ethylenically unsaturated mono- and dicarboxylic acids with C₂-C₃₀-aminoalcohols which have a primary or secondary amino group, primary amides of α,β-ethylenically unsaturated monocarboxylic acids, esters of vinyl alcohol and allyl alcohol with C₁-C₃₀-monocarboxylic acids, vinyl ethers, vinylaromatics, vinyl halides, vinylidene halides, C₁-C₈-monoolefins, nonaromatic hydrocarbons with at least two conjugated double bonds and mixtures thereof.
